(19) United States
(12) Patent Application Publication
(10) Pub. No.: US 2016/0175611 A1
(43) Pub. Date: Jun. 23, 2016

(54) DEVICE FOR WIRELESSLY STIMULATING BODY USING LIGHT

(71) Applicant: RESEARCH & BUSINESS FOUNDATION SUNGKYUNKWAN UNIVERSITY, Suwon-si (KR)

(72) Inventors: Tae-Il Kim, Seoul (KR); Bong-Kiun Kaang, Seoul (KR); Byeong-Hak Park, Seosan-si (KR); Woo-Jin Jung, Seoul (KR)

(73) Assignee: RESEARCH & BUSINESS FOUNDATION SUNGKYUNKWAN UNIVERSITY, Suwon-si (KR)

(21) Appl. No.: 14/978,257

(22) Filed: Dec. 22, 2015

(30) Foreign Application Priority Data
Dec. 22, 2014 (KR) 10-2014-0186032

(51) Int. Cl. A61N 5/06

(52) U.S. Cl. CPC A61N 5/0622; A61N 2005/0643; A61N 2005/0667

(57) ABSTRACT

A device for stimulating a body using light is disclosed. The device for stimulating a body using light includes a transmission light generator capable of generating light; and a body organ stimulator that generates stimulating light for stimulating a body organ when transmission light received from the transmission light generator includes light of a preset wavelength band.

### CROSS-REFERENCE TO RELATED APPLICATION

This application claims the benefit under 35 USC 119(a) of Korean Patent Application No. 10-2014-0186032, filed on Dec. 22, 2014, the entire disclosure of which is incorporated herein by reference for all purposes.

### BACKGROUND

1. Field of the Invention

The present invention relates to a device for wirelessly stimulating a body using light, and more particularly, a wirelessly operated apparatus for irradiating a body organ with light and examining a response of the body.

2. Discussion of Related Art

Optogenetics is an interdisciplinary academic and technological area formed by fusing optics and genetics, and was pioneered and advanced in the laboratory of Professor Deisseroth at Stanford University. Optogenetics is a cutting edge technology for controlling nerve cells with light, where genes of a light sensitive protein such as Channelrhodopsin2 (ChR2: responds to blue emission from GaN) found in cell membranes are inserted into nerve cells of a test animal using a virus vector and the nerve cells are stimulated according to the wavelength of the light and are controlled by being activated or suppressed.

In order to control nerve cells with light, both wired communication techniques and wireless communication techniques are being utilized. In the case of wired communication techniques, for example, an optical stimulation delivered through an optical fiber or an electrical stimulation of a body using a common electrical wire is used, and since these wired communication techniques use an optical fiber or a wire, there is a problem that tangling may occur when a plurality of lines are connected to a plurality of bodies. When lines get tangled, there is a concern that, lines themselves being damaged, and a body may also be damaged by the tangled wires.

Also, when an optical fiber is used for optical stimulation, in order to directly couple the optical fiber to a nerve of the body, a bulky and heavy cannular made of steel is required, and thus there is a problem in that a target organ (for example, a brain) of the body for optical stimulation connected to the cannular may come under a stress. Also, because the cannular has the large bulk and weight, there is also a problem in that it is difficult to apply the cannular to a small body such as a mouse is.

To overcome these problems, a wireless body stimulation device utilizing an inductive coupling technique was developed at MIT and published in a journal, but the wireless body stimulation technique has a limit in that it can operate only in a short range, and thus there is a problem in that it is difficult to apply the technique by various schemes without limit of range. Also, for simultaneously stimulating a plurality of bodies using a plurality of wireless body stimulation devices using light, different electric fields need to be applied to each wireless body stimulation device, but there is a problem in that such a simultaneous application of different electric fields to each wireless body stimulating device is difficult in practice.

In order to overcome the above problems, as wireless communication techniques, for example, a body stimulating device using a radio frequency technique is being currently developed, but even in this case, a problem still exists that it is difficult to simultaneously apply optical stimulus to a plurality of bodies using a plurality of wireless stimulating devices, and a problem also exists that errors may be occurred due to the polarization characteristics of the radio frequency waveform.

Also, a wireless body stimulation device to which the wireless communication technique has been applied has problems of bulkiness, high cost, and requiring a large electric power for operation, and since such a device generates a strong electric or magnetic field when operated, there is a problem in that the device cannot directly be used on bodies sensitive to an electric or magnetic field.

Also, since wireless communication techniques are influenced by a great number of external noise sources when used, there is a problem in that noise ratio to a signal is large.

### SUMMARY OF THE INVENTION

The present invention is directed to providing a device for stimulating a body using light which is capable of being wirelessly controlled using light as a medium and light for stimulate a body organ is generated only when the body organ is irradiated with light with a preset wavelength band to stimulate the body organ.

According to an aspect of the present invention, there is provided a device for stimulating a body using light including a transmission light generator capable of generating light; a body organ stimulator that generates a stimulating light for stimulating the body organ when transmission light received from the transmission light generator includes a preset wavelength band.

The transmission light generator may perform functions that are similar to a remote controller, that is, a means for controlling the body organ stimulator using light.

The transmission light generator may be configured to emit transmission light with various wavelength bands. For example, two or more light sources may be configured to emit transmission light with various wavelength bands, and light with a specific wavelength band may be emitted by providing a filter in the light sources in a broad wavelength band such as white light.

The body organ stimulator may be configured as a device that is detachable or capable of being inserted. As an example, in the case of the detachable device, examining a reaction of a treatment subject (for example, a person or a mouse) to an optical stimulus may be possible when the body organ stimulator is configured to be attached to the treatment subject to irradiate skin or inside of the skin of with light. In another example, in the case of being capable of being inserted, examining of an optogenetic response or a response to an electrical stimulation that occurs inside a body may be possible when the body organ stimulator is configured to be inserted into the body to irradiate deep site of a brain with light.

The body organ stimulator may include a stimulating light emitter that generates the stimulating light for stimulating the body organ, a stimulator power supply that supplies electrical power to the stimulating light emitter, and a transmission light receiver that receives the transmission light, wherein the stimulator power supply may supply electrical power to the stimulating light emitter when the transmission light received from the transmission light generator includes a preset wavelength.

The stimulating light emitter, the stimulator power supply, and the transmission light receiver may be attached to a subject receiving the optical stimulus. For example, the device for stimulating a body using light is attached to a subject such as a mouse, and when remotely controlling the operation and the intensity of the optical stimulus, movement of the subject is controllable.

The transmission light receiver may be a photo transistor.

The transmission light generator may have a filter which filters only light with a specific wavelength band. For example, light with a desired wavelength may be generated from one light source by using the transmission light generator light emitter that generates white light and by providing the filter.

The transmission light receiver may have a filter which filters only light with a specific wavelength band. For example, when using a photo transistor that responds to light with a broad wavelength band such as white light, by providing the filter, light with a wavelength out of the filtering is blocked, and when the light with the filtered wavelength is used, the photo transistor may produce a responsive result.

The body organ includes genes responsive to the stimulating light, for example, Channelrhodopsin genes.

The transmission light emitter of the transmission light generator may be disposed above a subject receiving the optical stimulus, and a transmission light receiver attached to the subject may be exposed and attached on a top surface of the subject to be capable of receiving light of the transmission light emitter.

The transmission light receiver may be configured such that an intensity of the stimulating light is controlled according to the transmission light. The photo transistor may strongly respond to a strong transmission light, strong current flows in a circuit of the body organ stimulator, and thus a strong stimulating light can be emitted.

The present invention can allow the effective irradiation of light with a body organ through the body organ stimulator without physical and location constraints since the body organ stimulator is wirelessly irradiated with the light generated by the transmission light generator.

According to the present invention described above, since the body organ stimulator is wirelessly irradiated with the light generated in the transmission light generator, it is possible to irradiate the body organ with light through the body organ stimulator without limitation.

In addition, according to the present invention, it is possible to selectively stimulate a specific body even when a plurality of bodies exist at a same site, by using a plurality of devices for stimulating a body using light.

The present invention can irradiate the body organ with light, therefor it can be effectively applied in numerous biological fields that utilize light, such as optogenetics and neuroscience, etc.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is an illustration for explaining a device for stimulating a body using light according to an embodiment of the present invention;

FIG. 2 is a graph showing a measurement of amounts of a current flowing in a body organ stimulator according to an embodiment of the present invention according to time;

FIG. 3 is an illustration for explaining a device for stimulating a body using light according to another embodiment of the present invention;

FIG. 4A and FIG. 4B are illustrations for explaining that the body organ stimulator according to an embodiment of the present invention is applied to a mouse;

FIG. 5 is an illustration for explaining that a specific mouse among a plurality of mice can be stimulated using light by using a plurality of the devices for stimulating a body using light.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that the exemplary embodiments are not in any way meant to be limiting and that various modifications and substitutions by equivalents may be made without departing from the spirit and scope of the invention.

Hereinafter, preferred embodiments according to the present invention will be explained with reference to the accompanying drawings. In the drawings, like numerals denote like elements.

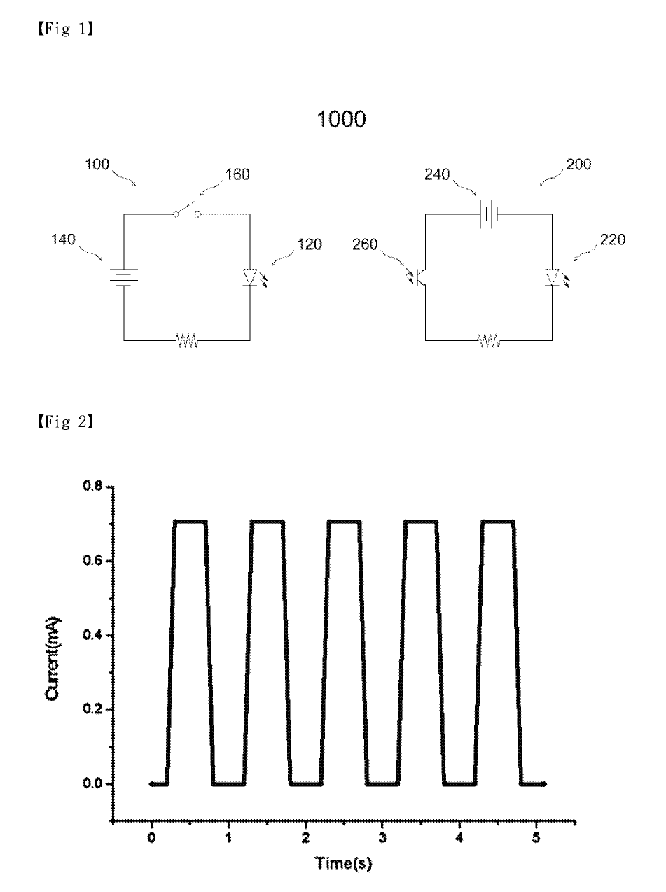

FIG. 1 is an illustration for explaining a device for stimulating a body using light according to an embodiment of the present invention.

Referring to FIG. 1, the device for stimulating a body using light 1000 according to an embodiment of the present invention includes a transmission light generator 100 and a body organ stimulator 200.

The transmission light generator 100 is a device capable of generating light with a specific wavelength band or a plurality of wavelength bands, and may include a transmission light emitter 120, a transmitter power supply 140, and a switch 160.

For example, the transmission light generator 100 may be adjusted to generate light with various wavelengths within the solar spectral band or only light with a specific wavelength band.

The transmission light emitter 120 may be a light emitting device such as a light-emitting diode (LED) capable of generating light with a specific wavelength band. For example an LED generating white light, an LED generating red light, an LED generating green light, and an LED generating blue light, etc. may be used as the transmission light emitter 120.

The transmission light emitter 120 may be configured to generate light with a desired specific wavelength band by using a color filter or a wavelength filter in one transmission light emitter 120, or to generate light with a desired specific wavelength band by using two or more transmission light emitters 120 each generating transmission lights in different wavelength bands,.

The transmitter power supply 140 may supply electrical power to the transmission light emitter 120. As an example, the transmitter power supply 140 may use a replaceable primary cell and a secondary cell, however as long as power may be supplied to the transmission light emitter 120, the transmitter power supply 140 is not limited to the primary cell and the secondary cell.

The switch 160 may control the supply of electrical power supplied by the transmitter power supply 140. As an example, by closing the switch, electrical power may be supplied from the transmitter power supply 140 to the transmission light emitter 120, and by opening the switch, electrical power supplied to the transmission light emitter 120 may be shut off from the transmitter power supply 140. As a switch, a transistor or the like which is a general switching element can be used, but a type of the switch is not limited thereto.

The body organ stimulator 200 is a device that generates stimulating light for stimulating the body organ when irradiated with the transmission light from the transmission light generator 100 and when the transmission light is light with a preset wavelength band. The body organ stimulator 200 includes a stimulating light emitter 220, a stimulator power supply 240, and a transmission light receiver 260.

The transmission light receiver 260 is configured as a photo transistor, and has a configuration such as a switch in which a current flows in a circuit of the body organ stimulator 200 by responding to a transmission light with a preset specific wavelength band. For example, when the body organ stimulator 200 includes a photo transistor configured to respond to light with green wavelength, if the transmission light that includes a green wavelength band is irradiated on the transmission light receiver 260, a switch in the circuit of the body organ stimulator 200 turns on, the current flows and the stimulating light emitter 220 emits light.

The body organ includes genes such as genes of Channelrhodopsin that responds to the light of the stimulating light emitter 220 so that the stimulating light of the body organ stimulator 200 may be intended to be used in optogenetics.

The stimulating light emitter 220 may generate stimulating light which stimulates the body organ. For example, an LED may be used as the stimulating light emitter 220, and an LED capable of generating light with a specific wavelength band to stimulate the body organ is preferable. For example, when the body organ stimulator 200 is applied to optogenetics, when the stimulating light emitter 220 generates light with a specific wavelength band so that the body organ that responds to the light with a specific wavelength band is irradiated with the generated light, the body organ may respond to the light with a specific wavelength band. The stimulating light emitter 220 may be changed depending on the type of light responsive genes, and the stimulating light emitter 220 may use a blue or green light emitter primarily used in optogenetics.

The stimulator power supply 240 may supply electrical power to the stimulating light emitter 220. As an example, the stimulator power supply 240 may use a replaceable primary cell and secondary cell, however as long as electrical power may be supplied to the stimulating light emitter 220, the stimulator power supply 240 is not limited to the primary cell and the secondary cell.

The transmission light receiver 260 causes the stimulator power supply 240 to supply electrical power to the stimulating light emitter 220 when irradiated with the light with a preset wavelength band from the transmission light generator 100. The transmission light receiver 260 may use an optoelectronic switch so that the transmission light receiver 260 operates depending on whether or not irradiated with light with a preset wavelength band. The transmission light receiver 260, for example, may be a photo transistor. The photo transistor is a transistor that allows a current to flow when irradiated with light with a specific wavelength band, and as such, when the transmission light receiver 260 is irradiated with the light with a specific wavelength band, the body organ stimulator 200 forms a closed circuit such that the stimulating light emitter 220 receives electrical power from the stimulator power supply 240, whereas on the other hand, when the transmission light receiver 260 is not irradiated with the light with a specific wavelength band, the body organ stimulator 200 forms an open circuit such that the stimulating light emitter 220 does not receive electrical power from the stimulator power supply 240, and thus the transmission light receiver 260 may serve as a switch depending on whether the transmission light receiver 260 is irradiated with light.

According to the present invention, a plurality of body organ stimulators 200 may be variably controlled. For example, when a white light with various wavelength bands is generated by the transmission light emitter 120, the body organ stimulator designed to operate in different wavelength bands may be operated. Also, when the transmission light including green and blue wavelength is emitted, the body organ stimulator with a photo transistor responsive to red light will not operate, rather, only the photo transistors responsive to green and blue light will operate.

Also, when the photo transistor is used as the transmission light receiver 260, the amount of the current flowing in the body organ stimulator 200 may be adjusted by the intensity of the irradiated light, and the amount of the current flowing in body organ stimulator 200 may be adjusted by controlling the intensity of the light generated by the transmission light generator 100 or the distance between the transmission light generator 100 and the body organ stimulator 200,. Also, by installing a variable resistor or other types of electrical circuits to adjust the amount of the current, the amount of the current flowing in the body organ stimulator 200 may be adjusted.

When a photo transistor is used as the transmission light receiver 260, the following experiments were performed to confirm that the amount of the current flowing in the body organ stimulator 200 is adjusted by the intensity of the irradiated light.

Experimental Example

The distance between the transmission light generator 100 and the body organ stimulator 200 according to the embodiment of the present invention was set to 33.5 cm, then the transmission light receiver 260 of the body organ stimulator 200 was irradiated with light generated by the transmission light generator 100, and the light generated by the transmission light generator 100 was pulse modulated to approximately 1 Hz.

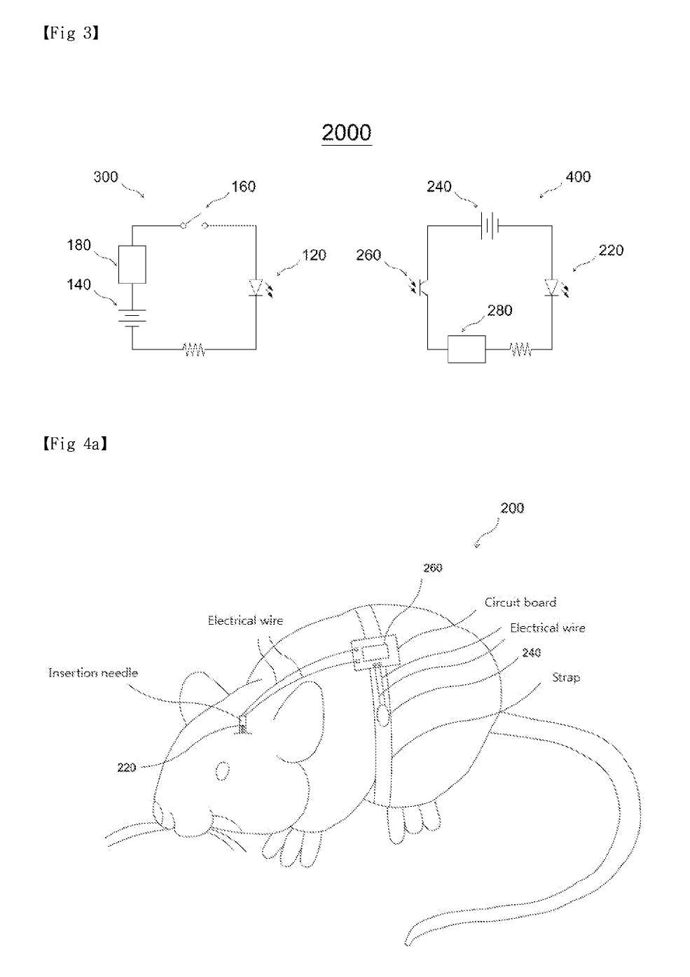

FIG. 2 is a graph showing a measurement of amounts of the current flowing in the body organ stimulator according to the embodiment of the present invention according to time.

Referring to FIG. 2, the current flowing in the body organ stimulator 200 was confirmed to have an amount of about 0.7 mA and a periodic pulse form according to time. By this, it was possible to confirm that the current flowing in the body organ stimulator 200 was consistently controllable by controlling the light generated by the transmission light generator 100.

FIG. 3 is an illustration for explaining a device for stimulating a body using light according to another embodiment of the present invention.

Referring to FIG. 3, a device for stimulating a body using light 2000 according to another embodiment of the present invention may include a transmission light generator 300 and a body organ stimulator 400. In the transmission light generator 300 illustrated in FIG. 3, the transmission light generator 100 illustrated in FIG. 1 further includes a first connector 180, and in the body organ stimulator 400 illustrated in FIG. 3, the body organ stimulator 200 illustrated in FIG. 1 further includes a second connector 280. Hereafter, the detailed explanations for FIG. 1 that are applicable to FIG. 3 will be omitted, and instead the first connector 180 and the second connector 280 will be explained in detail.

The transmission light generator 300 may include the transmission light emitter 120, the transmitter power supply 140, the first switch 160, and the first connector 180. The first connector 180 may be configured to be electrically connectable to an external electronic device. As an example, the first connector 180 may include electrodes capable of being connected to an external electronic circuit. The application range of the transmission light generator 300 may be expanded according to the characteristics of an electronic device connectable via the first connector 180.

For example, the application range of the transmission light generator 300 may be expanded by connecting the compatible circuit configured to be compatible with other electronic devices to the other electronic devices via the first connector 180, and when a first amplifier (not shown) is connected to the first connector 180, the electrical power supplied by the transmitter power supply 140 may be amplified, and as an example, an op-amp, etc. may be used as the first amplifier.

The body organ stimulator 400 may include the stimulating light emitter 220, the stimulator power supply 240, the transmission light receiver 260, and the second connector 280. The second connector 280 may be configured to be electrically connectable to an external electronic device. As an example, the second connector 280 may include electrodes capable of being connected to an external electronic circuit. The application range of the body organ stimulator 400 may be expanded according to the characteristics of an electronic device connectable via the second connector 280.

For example, the application range of the body organ stimulator 400 may be expanded by connecting the compatible circuit configured to be compatible with other electronic devices to the other electronic devices via the second connector 280, and when a second amplifier (not shown) is connected to the second connector 280, the electrical power supplied by the stimulator power supply 240 may be amplified, and as an example, an op-amp, etc. may be used as the second amplifier.

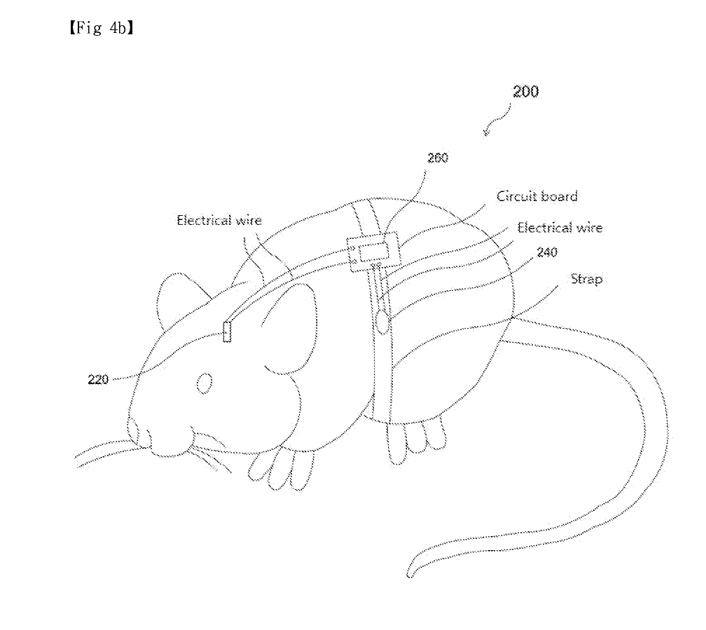

FIG. 4A and FIG. 4B are illustrations for explaining that the body organ stimulator according to an embodiment of the present invention is applied to a mouse.

Referring to FIG. 4A, the transmission light receiver 260 is disposed on a printed circuit board (PCB), and the stimulating light emitter 220 and the stimulator power supply 240 are each electrically connected to the printed circuit board by an electrical wire. The stimulating light emitter 220 is attached to an insertion needle so as to be inserted into a brain of a mouse. The stimulating light emitter 220 and the insertion needle may be inserted into the brain of the mouse together. Also, a strap for using the body organ stimulator 200 to the mouse is provided, and the stimulator power supply 240 is attached to the mouse.

Referring to FIG. 4B, unlike the explanation for FIG. 4A, the stimulating light emitter 220 is attached to a portion of the mouse head. This is because when the stimulating light emitter 220 emits light, the emitted light may penetrate the skin and stimulate the brain. Accordingly, an insertion needle for inserting the stimulating light emitter 220 into the brain of the mouse is not needed. Other than these points, remaining descriptions are the same as those of FIG. 4A and will be omitted.

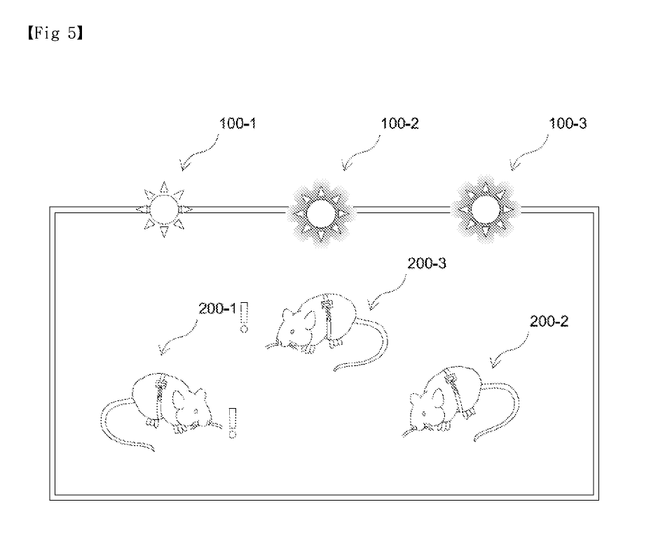

FIG. 5 is an illustration for explaining that a specific mouse among a plurality of mice can be stimulated using light by using a plurality of the devices for stimulating a body using light.

Referring to FIG. 5, three transmission light generators 100-1, 100-2, and 100-3, and three body organ stimulators 200-1, 200-2, and 200-3 are provided. As an example, the transmission light generators 100-1, 100-2, and 100-3 may be configured to be disposed above bodies (subjects) to be stimulated using light, and each of transmission light receivers of the body organ stimulators 2001-1, 200-2, and 200-3 may be mounted to be exposed on a top surface of each subjects of stimulation using light to receive light generated from the transmission light generators 100-1, 100-2, and 100-3.

Each of transmission light generators 100-1, 100-2, and 100-3 includes a first transmission light generator 100-1 emitting red light, a second transmission light generator 100-2 emitting blue light, and a third transmission light generator 100-3 emitting green light.

Each of body organ stimulators 200-1, 200-2, and 200-3 includes a first body organ stimulator 200-1 that generates a stimulating light in response to red light, a second body organ stimulator 200-2 that generates a stimulating light in response to blue light, and a third body organ stimulator 200-3 that generates a stimulating in response to green light.

When each of the second transmission light generator 100-2 and the third transmission light generator 100-3 generate light simultaneously, each of the second body organ stimulator 200-2 and the third body organ stimulator 200-3 may operate to generate a stimulating light to stimulate each mouse. However, because the transmission light receiver of the third body organ stimulator 200-3 does not respond to the red light generated by the first transmission light generator 100-1, the stimulating light of the third body organ stimulator is not emitted, and thus the mouse is not stimulated. As explained, when a plurality of the devices for stimulating a body with light according to an embodiment of the present invention is used, it is possible to selectively stimulate a specific body even when a plurality of bodies exists in a same site.

What is claimed is:

1. A device for stimulating a body using light, comprising:
a transmission light generator capable of generating light; and
a body organ stimulator that generates stimulating light for stimulating a body organ when transmission light received from the transmission light generator includes a preset wavelength band.

2. The device of claim 1, wherein the body organ stimulator includes:
a stimulating light emitter that generates the stimulating light which stimulates a body organ;
a stimulator power supply that supplies electrical power to the stimulating light emitter; and
a transmission light receiver that receives the transmission light;
wherein the stimulator power supply supplies electrical power to the stimulating light emitter when the transmission light received from the transmission light generator includes a preset wavelength.

3. The device of claim 2, wherein the stimulating light emitter, the stimulator power supply, and the transmission light receiver are attached to a subject receiving the optical stimulus.

4. The device of claim 3, wherein the transmission light receiver is a photo transistor.

5. The device of claim 1, wherein the transmission light generator has a filter which filters only light with a specific wavelength band.

6. The device of claim 2, wherein the transmission light receiver has a filter which filters only light with a specific wavelength band.

7. The device of claim 1, wherein the body organ includes genes responsive to the stimulating light.

8. The device of claim 7, wherein the genes include Channelrhodopsin genes.

9. The device of claim 1, wherein a transmission light emitter of the transmission light generator is disposed above a subject receiving the optical stimulus, and a transmission light receiver attached to the subject is exposed and attached on a top surface of the subject to be capable of receiving light of the transmission light emitter.

10. The device of claim 2, wherein the transmission light receiver is configured such that an intensity of the stimulating light is controlled according to the transmission light.
